(19) United States
(12) Patent Application Publication
(10) Pub. No.: US 2006/0058654 A1
(43) Pub. Date: Mar. 16, 2006

(54) System and method for providing a user interface for an ultrasound system

(76) Inventors: Gerois Di Marco, Waukesha, WI (US); Snehal Chandrakant Shah, Milwaukee, WI (US); John Edward Dwyer JR., Muskego, WI (US); Steven Charles Miller, Waukesha, WI (US)

Correspondence Address: Dean D. Small;Armstrong Teasdale LLP, Suite 2600, One Metropolitan Square, St. Louis, MO, 63102, US

(21) Appl. No.: 10/924,621

(22) Filed: Aug. 24, 2004

(51) Int. Cl. A61B 8/00

(52) U.S. Cl. 600/437

(57) ABSTRACT

A user interface for an ultrasound system is provided. The ultrasound system includes an ultrasound probe. The user interface includes at least one user actuable input member for controlling operation of an ultrasound system. The at least one user actuable input member is user configurable to control at least one operation of the ultrasound system. The user interface also has a controller including the at least one user actuable input member. The controller is removably attachable to the ultrasound probe.

### BACKGROUND OF THE INVENTION

The invention relates generally to ultrasound systems, and more particularly, to user interfaces for ultrasound systems.

Medical ultrasound systems may be used to examine and study, for example, anatomical structures, detect anomalies in tissues and measure blood flow within the body. Ultrasound systems typically include an ultrasound probe that is used to transmit pulses of ultrasound waves, known as imaging pulses, into the body. Acoustic echo signals are generated at interfaces in the body in response to these waves. These echo signals are received by the ultrasound probe and transformed into an electrical signal that is used to produce an image of the body part under examination. This image is displayed on a display device.

In typical ultrasound systems, while performing a scan, users typically have to go back and forth to an ultrasound scanner to adjust or control the change of a parameter of the ultrasound probe or the ultrasound system. These adjustments or control changes include, for example, gain adjustment, freezing frames for printing, transmitted-wave amplitude modification, and modification of other control parameters used to obtain an optimal setting for the diagnostic image. These ultrasound systems, however, provide limited flexibility and user-friendliness. The user normally has to look at the ultrasound image display unit and, at the same time, manipulate control parameters from the ultrasound scanner. This procedure makes it difficult for the user to efficiently scan and obtain ultrasound images. For example, when a desired frame is seen on the display that needs to be captured and printed, the user needs to freeze it from the ultrasound scanner. While doing so, there is lost scan time and the ultrasound probe also may get disturbed because of the user's movements, causing undesirable results. Further, typical ultrasound systems are not able to provide adequate flexibility when the user must use both hands. An example of such a case is when a user uses one hand for the probe, and the other to manipulate the scanning anatomy or perform interventional procedures. An example of patient manipulation is applying and removing pressure to stimulate venous flow. Interventional procedures include biopsies or therapeutic seed implants.

Further, ultrasound systems in a multi-user environment do not provide an easy and practical way to have each user settings programmed on each ultrasound system. For example, a user in a hospital with many ultrasound systems typically must adjust control settings and parameters on each ultrasound system before and during an ultrasound scan. Any changes to settings are stored on the current ultrasound system and cannot be easily transferred to another ultrasound system.

Thus, known ultrasound systems and methods for providing ultrasound imaging provide limited flexibility and user-friendliness.

### BRIEF DESCRIPTION OF THE INVENTION

In one exemplary embodiment, a user interface for an ultrasound system is provided. The ultrasound system includes an ultrasound probe. The user interface includes at least one user actuable input member for controlling operation of the ultrasound system. The at least one user actuable input member is user configurable to control at least one operation of the ultrasound system. The user interface also has a controller including the at least one user actuable input member. The controller is removably attachable to the ultrasound probe.

In another exemplary embodiment, a controller for an ultrasound system having an ultrasound probe is provided. The controller includes means for receiving at least one user input for controlling the ultrasound system. The controller also includes means for removably attaching the means for receiving to the ultrasound probe. The controller further includes means for communicating the at least one received user input to the ultrasound system for controlling the ultrasound system.

In yet another exemplary embodiment, a method for controlling an ultrasound system having an ultrasound probe is provided. The method includes receiving user inputs at an ultrasound probe. The user inputs correspond to control functions for controlling the ultrasound system and are configurable by a user. The method further includes communicating the user input to ultrasound system to control operation of the ultrasound system.

### BRIEF DESCRIPTION OF THE DRAWINGS

FIG. 1 is a block diagram of a user interface for an ultrasound system in accordance with an exemplary embodiment of the invention.

FIG. 2 is a top plan view of a user interface attached to an ultrasound probe in accordance with an exemplary embodiment of the invention.

FIG. 3 is a side elevation view of a user interface attached to an ultrasound probe in accordance with an exemplary embodiment of the invention.

FIG. 4 is a block diagram illustrating communication between a user interface and an ultrasound scanner connected to an ultrasound probe in accordance with an exemplary embodiment of the invention.

FIG. 5 is a block diagram of a controller in accordance with an exemplary embodiment of the invention.

FIG. 6 is a block diagram illustrating a user configuration stored in a memory in accordance with an exemplary embodiment of the invention.

FIG. 7 is a block diagram illustrating a set of user identifiers and associated user configurations stored in a memory in accordance with an exemplary embodiment of the invention.

FIG. 8 is a flowchart illustrating a method to control an ultrasound system in accordance with an exemplary embodiment of the invention.

### DETAILED DESCRIPTION OF THE INVENTION

Various embodiments of the invention provide a user interface and method for controlling an ultrasound system having an ultrasound probe. The user interface is operable at an ultrasound probe and the operations of the ultrasound probe or the ultrasound system may be controlled through the user interface attached to the ultrasound probe.

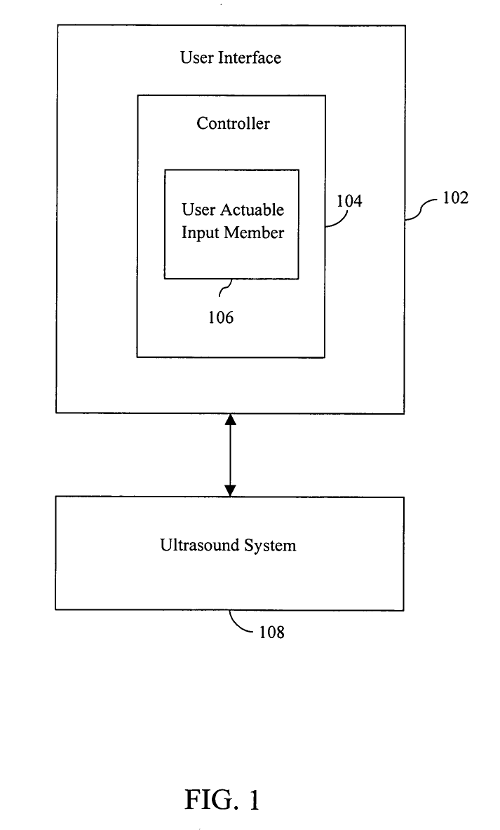

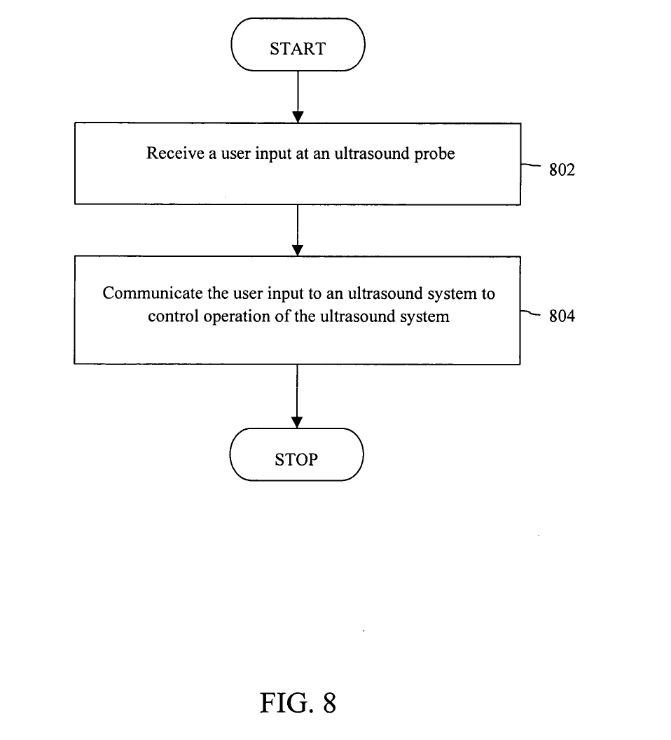

FIG. 1 is a block diagram of a user interface 102 for an ultrasound system in accordance with an exemplary embodiment of the invention. User interface 102 includes a controller 104 and at least one user actuable input member 106. User interface 102 is connected to an ultrasound system 108 via a wireless or wired connection. In one embodiment, the ultrasound system 108 includes an ultrasound probe (not shown). Controller 104 controls operations of ultrasound system 108. The control of operations of ultrasound system 108 by controller 104 is described in detail in connection with FIG. 8. User actuable input member 106 is provided as part of controller 104 (e.g., integrated therewith), and more than one user actuable input member 106 may be provided as part controller 104 as desired or needed. User actuable input member 106 may be, for example, a button, switch, knob, dial, wheel, touch pad, joystick and slide, among others.

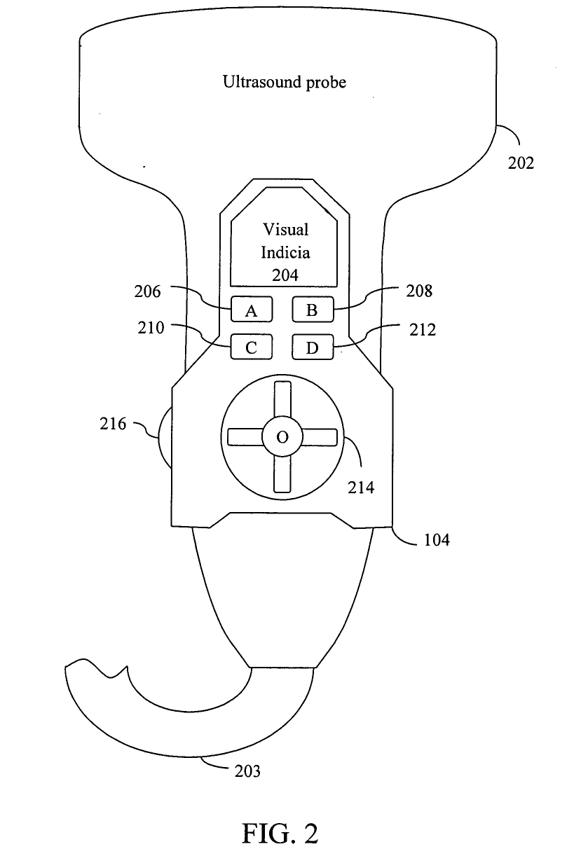

In an exemplary embodiment of the invention, the operations of ultrasound system 108 and ultrasound probe 202 (shown in FIG. 2), which may be connected to ultrasound system 108, may be controlled by user interface 102 and include, for example, image capture operations, such as freeze, print, and cine-loop, scanning mode controls, such as switching between 2D imaging and Doppler or M-mode in a duplex display, acquisition trigger controls, such as extended field of view or 3D acquisition, trigger, image optimization controls, such as automatic image optimization, user presets, frequency, application, gain, dynamic range, edge enhance, depth, display depth, transmit focus controls, among others.

In one exemplary embodiment of the invention, ultrasound system is an ultrasound scanner. In this embodiment, user actuable input member 106 controls at least one scanning operation of the ultrasound scanner. In another embodiment, user actuable input member 106 controls at least one non-scanning operation of the ultrasound scanner. Examples of non-scanning operations of ultrasound system 108 include identification and authentication of a user. In an exemplary embodiment of the invention, for example, a user may be identified and authenticated by a login id and a password.

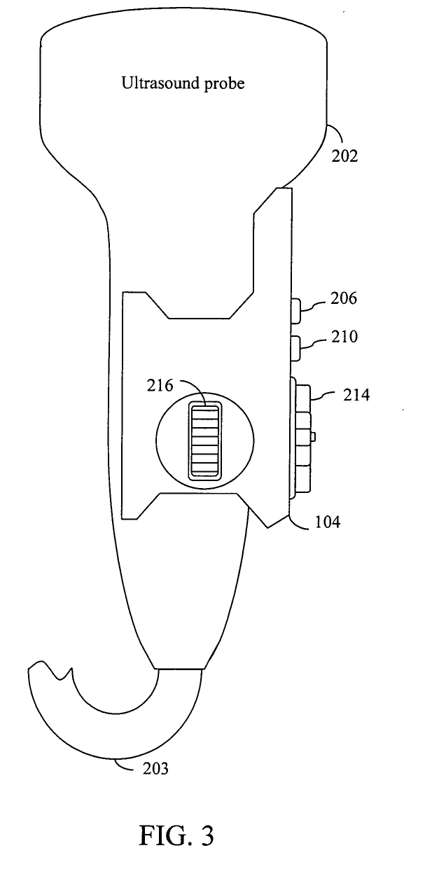

FIG. 2 is a top plan view of user interface 102 attached to an ultrasound probe in accordance with an exemplary embodiment of the invention. FIG. 3 is a side elevation view of user interface 102 attached to the ultrasound probe in accordance with an embodiment of the invention. User interface 102 is removably attachable to ultrasound probe 202.

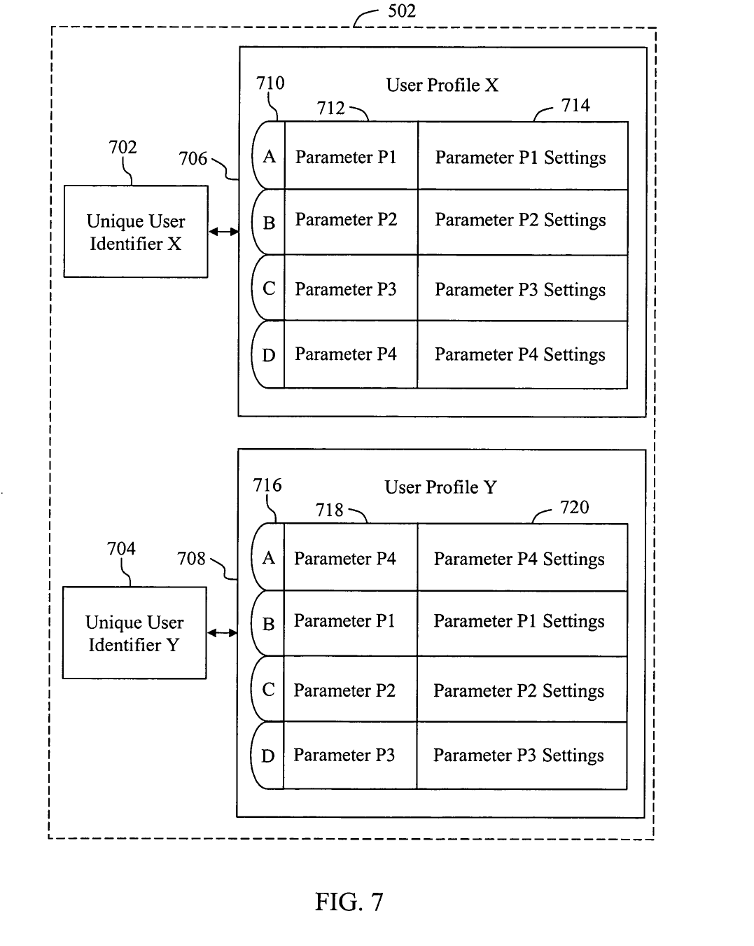

In various embodiments, body of controller 104 includes arms configured to extend around a portion of ultrasound probe 202 to removably attach user interface 102 to ultrasound probe 202. Body of controller 104 includes a top surface with an extension, which in one embodiment includes at least one user actuable input member 106 provided as a part of the extension. In another embodiment, controller 104 has a C like shape to attach to ultrasound probe 202. User actuable input member 106 is reconfigurable, for example, to control operation of ultrasound probe 202 or ultrasound system 108, and operable using user actuable input member 106, which can be reconfigured and changed. The reconfiguration of user actuable input member 106 is described in detail in connection with FIG. 7.

Ultrasound probe 202 includes a cable 203. Cable 203 connects ultrasound probe 202 to ultrasound system 108, for example an ultrasound scanner (not shown in FIG. 2). Controller 104 includes, for example, user actuable input members 206, 208, 210, 212, 214 and 216, and a visual indicia 204. In this embodiment, user actuable input members 206, 208, 210 and 212 are buttons that are pressed to actuate an operation of ultrasound probe 202 or ultrasound system 108. User actuable input member 214 is a rocker type switch that is used to actuate another operation of ultrasound system 108 or ultrasound probe 202 and modify it simultaneously. For example, user actuable input member 214 may be used to actuate a gain adjustment operation and then increase and decrease the gain. User actuable input member 216 in this embodiment is a scroll wheel that can be turned to actuate and, at the same time, make adjustments to an operation of ultrasound system 108 or ultrasound probe 202. User actuable input member 216 is positioned, for example, on a right side of controller 104 to facilitate a right-handed user to turn user actuable input member 216 using the thumb. In another embodiment, user actuable input member 216 is positioned on a left side of controller 104 to facilitate a left-handed user. Controller 104 is configured to provide visual indicia 204 to associate controller 104 with a user (e.g., user name or ID number).

In another embodiment, a scroll wheel may be provided on the upper surface of controller 104. Controller 104 may include other or different types of user actuable input members to provide a control of different operators as desired or needed.

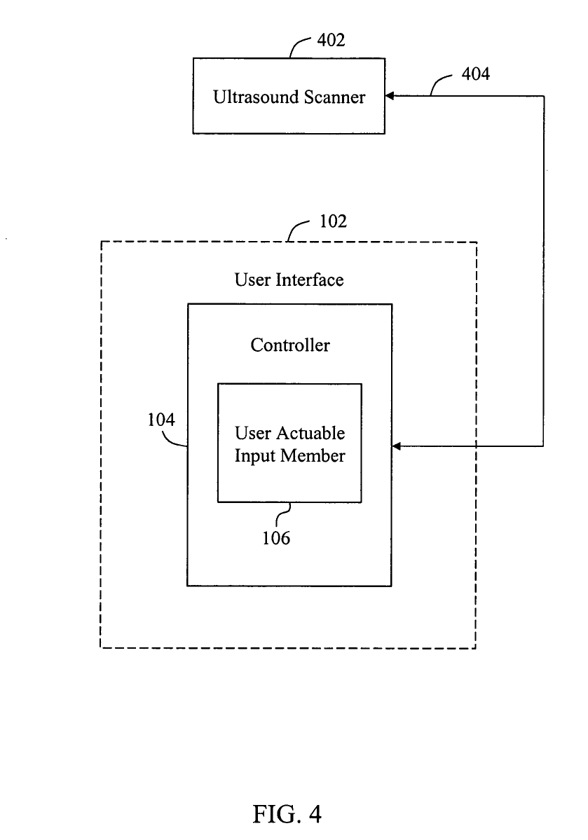

To control the operation of ultrasound system 108 or ultrasound probe 202, controller 104 communicates with ultrasound system 108, for example, an ultrasound scanner. In an exemplary embodiment of the invention, ultrasound system 108 is an ultrasound scanner. FIG. 4 is a block diagram illustrating the communication between a controller and an ultrasound scanner connected to ultrasound probe 202 in accordance with an exemplary embodiment of the invention. An ultrasound scanner 402 is connected to controller 104 through a communication link 404. Communication link 404 is one of a wireless and wired connection. In one embodiment, controller 104 is configured to wirelessly communicate with ultrasound scanner 402 through communication link 404. Wireless communication between controller 104 and ultrasound scanner 402 may be provided, for example, using one of Bluetooth, RF, radio signals, wireless LANs, wireless networks, and infra red. In this embodiment, wireless transceivers (not shown) are provided in controller 104 and ultrasound scanner 402 to enable wireless communication as is known. In another embodiment, controller 104 is configured to communicate with ultrasound scanner 402 through a wired connection provided by communication link 404. The wired connection may be a wire separate from cable 203 that connects ultrasound probe 202 with ultrasound scanner 402. In another embodiment, cable 203 is used for communication between controller 104 and ultrasound scanner 402. Where communication is enabled with a cable separate from cable 203, the separate cable is clipped on or mounted onto cable 203. This facilitates the handling of the ultrasound probe with still one cable to maneuver for the user.

Once communication between controller 104 and ultrasound scanner 402 is established, user adjustable parameters, actuated by a user to control the operation of ultrasound probe 202 or ultrasound scanner 402 are communicated to ultrasound scanner 402 from controller 104. Ultrasound scanner 402 then performs a corresponding action or function to control the operations of ultrasound scanner 402 based on the received user input (e.g., user input relating to an adjustable parameter). User adjustable parameters to control the operation of ultrasound system 108 or ultrasound probe 202 are also adjustable on a control panel (not shown in FIG. 4) of ultrasound scanner 402.

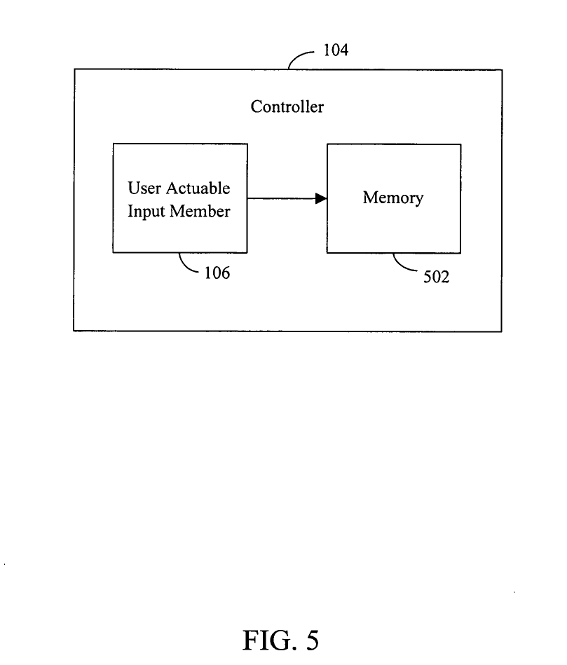

According to user habits, choices and practices, users may select and store a user profile in a memory (shown in FIG. 5) in controller 104. The user profile defines user predetermined settings for controlling ultrasound system 108 or ultrasound probe 202 using user actuable input members. For example, a user profile may include presets for imaging optimization controls like gray map, dynamic range, edge enhance, based on the context of the selected probe. In general, user profile includes user adjustable parameters associated with each user actuable input member and preset or predetermined system settings for that user.

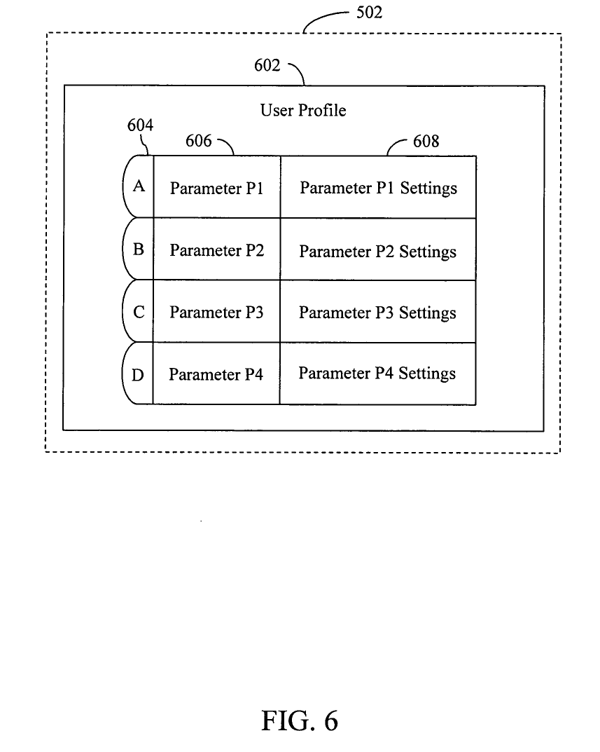

FIG. 5 is a block diagram of controller 104 in accordance with an exemplary embodiment of the invention. Controller 104 includes at least one user actuable input member 106 and a memory 502. Although FIG. 5 shows one user actuable input member 106, there may be a plurality of user actuable input members 106 included as part of controller 104. In another embodiment, memory 502 is located outside controller 104. Memory 502 may be one of a removable non-volatile memory device/card and plug-in drive. Memory 502 stores the user profile corresponding to user actuable input members 106 and any other user presets or predetermined system settings. The contents of the user profile are described in detail in conjunction with FIG. 6.

FIG. 6 is a block diagram illustrating a user profile stored in a memory 502 (shown in FIG. 5) inside controller 104 (shown in FIG. 1) in accordance with an exemplary embodiment of the invention. Memory 502 stores a user profile 602. User profile 602 includes the following columns: tag 604, parameter 606 and parameter setting 608. Column tag 604 stores a tag associated with a user actuable input member, for example, the element in the first row of column tag 604 contains ‘A’, which is the tag associated with user actuable input member 206 (as shown in FIG. 2). Column parameter 606 stores a user adjustable parameter. The user adjustable parameter corresponds to a user actuable input member, for example, the element in the first row of column parameter 606 contains ‘Parameter P1’, which is the user adjustable parameter corresponding to user actuable input member 206. ‘Parameter P1’ also defines a user configuration corresponding to user actuable input member 206 that corresponds to a control operation of ultrasound system 108 or ultrasound probe 202, for example, gain adjustment. Column parameter setting 608 stores a user predefined setting corresponding to user adjustable parameter stored in column parameter 606. For example, the element in the first row of column parameter setting 608 contains ‘Parameter P1 Settings’, which is a user predefined setting corresponding to ‘Parameter P1’ stored in the first row of column parameter 606. Each row in user profile 602 corresponds to a user actuable input member, for example, the first row corresponds to user actuable input member 206 (as shown in FIG. 2). Other known ways of storing user profile in memory 502 also may be used. Further, additional information corresponding to a particular user's presets or predetermined system settings for the ultrasound scanner may be stored (e.g., initial start-up settings). This additional information may include user presets for scanner parameters not directly accessible by user actuable input members, but stored into the memory in response to use activation of a “profile store” command via a user actuable input member 206 or the scanner user interface.

When a user actuates a user actuable input member, the corresponding user adjustable parameter and user predefined settings for that user adjustable parameter are communicated to ultrasound scanner 402 and the operations of ultrasound scanner 402 can then be controlled. For example, if the user actuates user actuable input member 206, ultrasound scanner 402 receives a signal that the user is requesting a control operation to control ultrasound scanner 402, corresponding to ‘Parameter P1’ with user-predefined settings ‘Parameter P1 Settings’. The scanner may also login the user to the system, providing access to HIPA protected data on the scanner.

In a multi-user environment, user profiles corresponding to a plurality of users may be stored in memory 502. Users may change their user profiles by reprogramming user actuable input members 106. Reprogramming of user actuable input members 106 is provided, for example, via a graphical user interface. In one embodiment, the graphical user interface is provided as part of ultrasound scanner 402. The graphical user interface may be configured, for example, as part of a screen. In the screen, a user can select from a list of control operations that may be assigned to a required user actuable input member. This screen provides, for example, a drop down menu, selection buttons, and other means of selection for reconfiguring the user actuable input members. The various control operations of ultrasound system 108 may be provided to the user for selection through the various selection means. The user also may input the settings and control operations that need to be assigned to each user actuable input member. The graphical user interface also provides a module for enabling control of the interface by a right-handed or a left-handed user. The graphical user interface also allows the user to select from a plurality of ultrasound probes 202 and control their operations connected to ultrasound scanner 402.

FIG. 7 is a block diagram illustrating a set of user identifiers and associated user profiles stored in a memory inside controller 104 (shown in FIG. 4) in accordance with an exemplary embodiment of the invention. To differentiate between various user profiles stored in memory 502, a unique user identifier is utilized for each profile. For example, in a multi-user environment, two users X and Y may use the same user interface 102 (as shown in FIG. 1). A unique user identifier X 702 (e.g., unique ID number) is associated with user X having user profile X 706. Both unique user identifier X 702 and user profile X 706 correspond to the user X. A unique user identifier Y 704 is associated with user Y having user profile Y 708. Both unique user identifier Y 704 and user profile Y 708 correspond to the user Y. Visual indicia 204 may be utilized to associate a user with controller 104 (shown in FIG. 1). Alternatively, and for example, color-coding of controller 104 may be provided.

User profiles are detected based on the unique user identifiers stored in memory 502. Ultrasound scanner 402 connected to ultrasound probe 202 automatically detects the user profile, logins the user, and configures controls of ultrasound scanner 402 to control operation of ultrasound probe 202, as well as the ultrasound scanner 402, based on the user profile. User X and user Y may re-program user actuable input members according to, for example, their requirements, and therefore, their respective user profiles 706 and 708 are different. For example, the first element in the first row of column parameter 606, ‘Parameter P1’, is different from the first element in the first row of column parameter 718, ‘Parameter P4’, (e.g., user X actuates ‘Parameter P1’ by actuating user actuable input member 206, while user Y actuates ‘Parameter P4’ by actuating user actuable input member 206). Thus for the same controller 104, control operations of ultrasound system 108 or ultrasound probe 202 are enabled for different users according to their requirements, which may include different control operations and/or associating different controls with the user actuable input members.

FIG. 8 is a flowchart illustrating a method to control ultrasound system 108 in accordance with an exemplary embodiment of the invention. At 802, a user input is received at ultrasound probe 202 via a user actuable input member. At 804, the user input is communicated to ultrasound system 108 so that the user can control the operation of ultrasound system 108 or ultrasound probe 202.

Input is received from the user after the user actuates a user actuable input member. The corresponding user configuration, associated with the user actuable input member being actuated, in the form of user adjustable parameters and associated user predefined settings are then communicated to ultrasound scanner via communication link 404. Ultrasound scanner 402 then controls the operation of ultrasound probe 202 accordingly.

Various embodiments of the invention may be used to control the operations of an ultrasound probe to generate ultrasound images. Remote operation of ultrasound probe 202 also may be enabled to perform scanning remotely. A user-friendly interface to control ultrasound system 108 or ultrasound probe 202 is provided that reduces the time required by a user to obtain an ultrasound image. Also, reprogramming of control buttons and associated functions are provided.

While the invention has been described in terms of various specific embodiments, those skilled in the art will recognize that the invention can be practiced with modification within the spirit and scope of the claims.

What is claimed is:

1. A user interface for an ultrasound system, the ultrasound system including an ultrasound probe, said user interface comprising:
at least one user actuable input member for controlling operation of the ultrasound system, the at least one user actuable input member being user configurable to control at least one operation of the ultrasound system; and
a controller including the at least one user actuable input member, the controller removably attachable to the ultrasound probe.

2. A user interface in accordance with claim 1 wherein at least one user actuable input member controls at least one scanning operation of the ultrasound system.

3. A user interface in accordance with claim 1 wherein at least one user actuable input member controls at least one non-scanning operation of the ultrasound system.

4. A user interface in accordance with claim 1 wherein the controller comprises a body having a shape corresponding to a portion of the ultrasound probe for removable attachment around at least part of the portion of the ultrasound probe.

5. A user interface in accordance with claim 1 wherein the at least one user actuable input member is reconfigurable.

6. A user interface in accordance with claim 1 wherein the controller is configured to wirelessly communicate with the ultrasound system.

7. A user interface in accordance with claim 1 wherein the controller is configured to communicate with the ultrasound system via a wired connection.

8. A user interface in accordance with claim 1 wherein the at least one user actuable input member is configurable to control at least one user adjustable parameter for controlling operation of the ultrasound system.

9. A user interface in accordance with claim 8 wherein the user adjustable parameter is adjustable on a control panel of the ultrasound system.

10. A user interface in accordance with claim 1 wherein the controller further comprises a memory for storing a user configuration corresponding to the at least one user actuable input member.

11. A user interface in accordance with claim 10 wherein the user configuration defines a control operation adjustable using the at least one user actuable input member.

12. A user interface in accordance with claim 11 wherein the control operation corresponds to a user adjustable parameter for controlling the operation of the ultrasound system.

13. A user interface in accordance with claim 1 wherein the controller further comprises a memory for storing a user profile corresponding to a plurality of user predefined settings for controlling operation of the ultrasound system.

14. A user interface in accordance with claim 13 wherein an ultrasound system connected to the ultrasound probe automatically detects the user profile and configures controls of the ultrasound system based on the user profile.

15. A user interface in accordance with claim 14 wherein the user profile is detected based on a unique user identifier stored in the memory.

16. A user interface in accordance with claim 15 wherein a plurality of user profiles are stored in the memory with a unique user identifier corresponding to each of the user profiles.

17. A user interface in accordance with claim 1 wherein the controller is configured to provide visual indicia to associate a controller with a user.

18. A user interface in accordance with claim 1 wherein the at least one user actuable input member is user programmable via a graphical user interface.

19. A user interface in accordance with claim 1 wherein the user actuable input member is configurable to control operation of a function of the ultrasound probe corresponding to a function available on an ultrasound control panel of an ultrasound scanner connected to the ultrasound probe.

20. A user interface in accordance with claim 1 wherein the user actuable input member comprises at least one of a button, switch, knob, dial and slide.

21. A user interface in accordance with claim 1 wherein the controller comprises a body having arms configured to extend around a portion of the ultrasound probe to removably attach the controller to ultrasound probe.

22. A user interface in accordance with claim 21 wherein the body further comprises a top surface with an extension having the at least one user actuable input member provided as part of the extension.

23. A controller for an ultrasound system having an ultrasound probe, said controller comprising:
means for receiving at least one user input for controlling the ultrasound system;
means for removably attaching the means for receiving to the ultrasound probe; and
means for communicating the at least one received user input to the ultrasound system for controlling the ultrasound system.

24. A controller in accordance with claim 21 further comprising means for storing user defined settings for controlling the ultrasound system.

25. A controller in accordance with claim 21 further comprising means for storing user programmable settings to define the control operation associated with the means for receiving.

26. A controller in accordance with claim 21 wherein the means for communicating comprise one of means for wirelessly communicating and means for communicating via a wired connection with the ultrasound system.

27. A method for controlling an ultrasound system having an ultrasound probe, said method comprising:
receiving a user input at the ultrasound probe, the user input corresponding to a control function for controlling the ultrasound system and configurable by a user; and
communicating the user input to an ultrasound scanner to control operation of the ultrasound system.
